(19) United States
(12) Patent Application Publication
(10) Pub. No.: US 2014/0276010 A1
(43) Pub. Date: Sep. 18, 2014

(54) Systems and Methods for Tracking Objects Using Magnetoresistance

(71) Applicant: General Electric Company, Schenectady, NY (US)

(72) Inventor: Peter Traneus Anderson, Andover, MA (US)

(73) Assignee: General Electric Company, Schenectady, NY (US)

(21) Appl. No.: 14/290,504

(22) Filed: May 29, 2014

Related U.S. Application Data
(63) Continuation-in-part of application No. 12/262,241, filed on Oct. 31, 2008.

(51) Int. Cl. A61B 19/00; A61M 25/01; A61B 1/00; A61B 5/06

(52) U.S. Cl. CPC A61B 19/54; A61B 5/062; A61M 25/0127; A61B 1/00158; A61B 2019/5458
USPC 600/424

(57) ABSTRACT

Tracking systems and associated methods for tracking the position and orientation of an object in the body using magnetoresistance are described. They include a position transponder located in an object to be tracked. The transponder contains a sensor coil configured to sense a voltage drop when an electromagnetic field is applied to the object containing the transponder, the electromagnetic field being applied from a transmitter external to the body and a magnetoresistive sensor coupled in series to the sensor coil via a single twisted pair or a coaxial cable. The transponder can transmit an output signal indicative of the position of the transponder within the object. The transponder can be part of a tracking system containing transmitters for applying the electromagnetic field and a signal processing unit for processing and optionally displaying the output signal. The tracking system can be used as part of a surgical navigation system.

### CROSS REFERENCE TO RELATED APPLICATION

This application is a continuation-in-part of U.S. patent application Ser. No. 12/262,241, filed on Oct. 31, 2008, the entire disclosure of which is incorporated herein by reference.

### FIELD

The invention generally relates to tracking systems and more particularly to methods and devices for tracking the position and orientation of an object in the body using magneto resistance (MR).

### BACKGROUND

Many surgical, diagnostic, therapeutic and prophylactic medical procedures require the placement of objects such as sensors, treatment units, tubes, catheters, implants and other objects within the body. In many instances, insertion of the object is for a limited time, such as during a surgery or catheterization. In other cases, objects such as feeding tubes or orthopedic implants are inserted for long-term use. A need exists for providing real-time information, for accurately determining the position and orientation of objects within a patient's body, while minimizing the use of X-ray imaging.

It is known to use tiny sensor coils as magnetic field transmitters and as magnetic field receivers, known as microcoils. Further, the use of magnetic field sensors in determining the position and orientation of an object inside the patient's body is known. Typically, the magnetic field sensor is located at the tip of a guidewire or a catheter and a plurality of leads connect the magnetic field sensor to an outside processing circuitry. The size of the magnetic field sensor located at the tip of the guidewire or the catheter is desired to be small and the number of leads connecting the magnetic field sensor to the outside processing circuitry is desired to be minimal.

Generally, a tracking system adapted for determining the position and orientation of an object employs at least one magnetic field sensor comprising a plurality of coils. A first coil provides five degrees of freedom (three position and two orientation coordinates) and a second coil provides the sixth degree of freedom.

### SUMMARY

This application describes tracking systems and associated methods for tracking the position and orientation of an object in the body using magnetoresistance (MR). The systems include a position transponder located in an object to be tracked. The transponder contains a sensor coil configured to sense a voltage drop when an electromagnetic field is applied to the object containing the transponder, the electromagnetic field being applied from a transmitter external to the body and a magnetoresistive device coupled in series to the sensor coil via a single twisted pair or a coaxial cable. The transponder can transmit an output signal indicative of the position of the transponder within the object. The transponder can be part of a tracking system containing transmitters for applying the electromagnetic field and a signal processing unit for processing and optionally displaying the output signal. The tracking system can be used as part of a surgical navigation system.

### BRIEF DESCRIPTION OF THE DRAWINGS

The following description can be better understood in light of the Figures, in which:

FIG. 1 shows a block diagram of a transponder in some embodiments;

FIG. 2 shows a block diagram of an tracking system using the transponder in other embodiments;

FIG. 3 shows a diagram of a tracking system used in conjunction with an imaging system in yet other embodiments; and

FIG. 4 shows a diagram depicting the method of tracking an object using a tracking system in some embodiments.

The Figures illustrate specific aspects of the systems and methods for tracking the position and orientation of an object in an object. Together with the following description, the Figures demonstrate and explain the principles of the methods and structures produced through these methods. In the drawings, the thickness of layers and regions are exaggerated for clarity. The same reference numerals in different drawings represent the same element, and thus their descriptions will not be repeated. As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c, etc.), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan would understand that the described systems and methods can be implemented and used without employing these specific details. Indeed, the described systems and methods can be placed into practice by modifying the illustrated devices and methods and can be used in conjunction with any other apparatus and techniques conventionally used in the industry. For example, while the description below focuses on systems and methods for tracking the position and orientation of an object in the body using MR, it can be combined with numerous other techniques and apparatus used for surgical navigation.

In the following detailed description, reference is made to the accompanying drawings that form a part hereof, and in which is shown by way of illustration specific embodiments, which, may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments.

In the embodiments shown in FIG. 1, a position transponder 105 for operation inside the body of a subject can be provided. The transponder 105 contains at least one electromagnetic sensor 106. The electromagnetic sensor 106 is composed of at least one microcoil 110 and at least one magnetoresistive (MR) device (acting as a magnetic field sensor) 115 that can be coupled in series to each other. One or more electromagnetic fields can be applied to the body in a vicinity of the transponder 105. The application of an electromagnetic field(s) can induce a voltage drop in each of the microcoil 110 and the MR device 115.

The transponder 105 also comprises a control unit 120 that can be coupled to the microcoil 110 and the MR device 115. In this configuration, the control unit 120 can generate an output signal indicative of the voltage drop induced at the microcoil 110 and the voltage drop induced at the MR device 115. The MR device 115 can be coupled to the microcoil 110 in series in a specific orientation relative to the microcoil 110 such that the MR device 115 can sense the electromagnetic field at a direction substantially perpendicular to the axis of the microcoil 110. The output signal is indicative of position and orientation of the transponder 105 inside the body. The control unit 120 can be configured to transmit the output signal to a signal processing unit positioned outside the body, such that the output signal is received by the signal processing unit for use in determining the position and orientation of the transponder 105.

In some embodiments, the transponder can be part of a tracking system. In these embodiments, the transponder 105 can be tracked against a plurality of transmitters and optionally a plurality of receivers. The plurality of transmitters emit at different respective frequencies including a second frequency, F1. The radio frequency driver can be configured to drive the MR device 115 of the transponder 105 with a sine wave at a first frequency, F0, as explained in detail below.

Accordingly, as shown in FIG. 2, a tracking system 200 for tracking an object (not shown) is provided. The tracking system 200 comprises a radio frequency driver 210 which is adapted to transmit a radiofrequency driving current to the object to be tracked. The transmitters 215 are adapted to generate electromagnetic fields at different respective frequencies in the vicinity of an object which contains the transponder 220 which contains microcoil 222 and MR device 224. The transponder 220 (which in some embodiments is similar to transponder 105) emits a frequency that is processed by signal processing unit 230.

In these embodiments, the plurality of transmitters 215 generate electromagnetic fields composed of a plurality of differently oriented field components each having a different known frequency in the range of about 2 to about 10 kHz. Each of these field components can be sensed by each of the microcoil 222 and the MR device 224 which each produce a signal comprising one or more frequency components having different amplitudes and phases depending on the relative distance and orientation of the particular microcoil 222 or the MR device 224 from the particular transmitter which transmits a particular frequency. The contributions of each of the transmitters 215 are used to solve a set of field equations, which are dependent upon the field form. Solving these equation sets produces the position and orientation of the transponder 220.

In some configurations, the transponder 220 can be about 2 to about 5 mm in length and about 2 to about 3 mm in outer diameter, enabling it to fit conveniently inside any desired object. The microcoil 222 can be optimized to receive and transmit high-frequency signals in the range of about 1 to about 3 MHZ, or any other frequencies at which the transmitters 215 generate the electromagnetic fields. Of course, other frequency ranges may be used as needed.

In some configurations, the microcoil 222 in the transponder 220 has an inner diameter of about 0.5 mm and has approximately 800 turns of about 16 micrometer diameter to provide an overall diameter in the range of about 1 to about 1.2 mm. In other configurations, these dimensions may vary over a considerable range as needed by the tracking system 200. The effective capture area of the microcoil 222 can be about 400 mm². The effective capture area is desired be made as large as feasible, consistent with the overall size requirements. Though the shape of the microcoil 222 used in some embodiment is cylindrical, other shapes can also be used depending on the geometry of the object (not shown). One non-limiting example of a microcoil 222 is the T30AA01 passive telecoil manufactured by the Sonion division of Pulse Engineer.

The electromagnetic fields produced by the transmitters 215 induce a voltage drop in the microcoil 222. The voltage drop at the microcoil 222 comprises a component at the second frequency, F1, the frequency of the electromagnetic fields produced by the transmitters 215. The voltage components are proportional to the strengths of the components of the respective magnetic fields produced by the transmitters 215 in a direction substantially parallel to the axis of the microcoil 222. Thus, the amplitudes of the voltages indicate the position and orientation of the microcoil 222 relative to the transmitters 215.

In some embodiments, the MR device 224 can be coupled to the microcoil 222 in series using one of a single twisted-pair and a coaxial cable. Thus, the MR device 224 can be adapted to sense the electromagnetic field at a direction substantially perpendicular to the axis of the microcoil 222. These embodiments are aimed at minimizing the field coupling between the microcoil 222 and the MR device 224.

One example of the MR device 224 is an extraordinary magneto resistance (EMR) device. Extraordinary magneto resistance (EMR) devices have been fabricated and characterized at various magnetic fields, operating temperatures, and current excitations. The extraordinary magneto resistance devices can be comprised of nonmagnetic high mobility semiconductors and low resistance metallic contacts and shunts. The resistance of the extraordinary magneto resistance device is modulated by magnetic fields due to the Lorentz force steering an electron current between a high resistance semiconductor and a low resistance metallic shunt.

In some configurations, the MR device 224 comprises a first portion where the resistance does not significantly change with the electromagnetic field. Therefore, the voltage drop at the MR device 224 comprises a component at the first frequency, F0, the frequency of the driving currents flowing through the transmitters 215.

In these configurations, the MR device 224 comprises a second portion where the electrical resistance of the MR device 224 varies responsive to the changing electromagnetic field. Following Ohm's law, V=IR, the MR device 224 develops a voltage drop that varies with the product of the applied electromagnetic field and the current through the MR device 224. As the driving current is at the first frequency, F0, with a zero direct current component, and the electromagnetic field is at the second frequency, F1, the voltage drop at the MR device 224 comprises components at the sum of the first frequency and the second frequency (F0+F1) and at the difference between the first frequency and the second frequency (F0−F1). As the voltage drops induced at the microcoil 222 and the MR device 224 due to the electromagnetic field are at different frequencies, the two voltage drops can then be distinguished.

The transponder 220 also contains a control unit 226. In some configurations, the control unit 226 is similar to control unit 120. The control unit 226 can be coupled to the microcoil 222 and the MR device 224 and contains suitable circuitry for reading the signals from the microcoil 222 and the MR device 224. On receiving the signals from the microcoil 222 and the MR device 224, the control unit 226 generates an output signal indicative of an amplitude of the voltage drop induced at the microcoil 222, an amplitude of the voltage drop induced at the MR device 224, and a phase of the voltage drop relative to a phase of the electromagnetic fields. The signal processing unit 230 can be adapted to determine the position and an orientation of the object responsive to the amplitude and the phase of the voltage drop indicated by the output signal.

The tracking system 200 also contains signal processing unit 230. Both analog and digital embodiments of signal processing are possible. The signal processing unit 230 can contain any number of components that can be used to process the signal(s) emitted from transponder 220. For example, such components may be configured to receive information or signals, process the signals, function as a controller, display information, and/or generate information or signals. Typically, the signal processing unit 230 may comprise one or more microprocessors.

The transponder 220 can be employed to provide all six position and orientation coordinates (X, Y, Z, yaw, pitch and roll) of the object in which it is contained. The single microcoil 222 shown in FIG. 2, in conjunction with the transmitters 215 (and optionally a plurality of receivers), enables the signal processing unit 230 to generate three dimensions of position and two dimensions of orientation information. The third dimension of orientation (typically the rotation of the object about its longitudinal axis, known as roll) can be inferred from the MR device 224. Although the signal from the MR device 224 can be smaller than the signal from the microcoil 222, the signal from the MR device 224 can be large enough to provide the roll information.

In some embodiments, the information can be obtained using a single microcoil 222 coupled with a single MR device 224 and can be used to determine the position and orientation of an object such as a medical device or medical instrument. In other embodiments, the transponder 220 may comprise more than one set of microcoils or MR devices that will provide sufficient parameters to determine the configuration of the object relative to a reference frame. As well, one or more MR devices can be used, along with one or more microcoils to obtain six position and orientation coordinates. For example, a plurality of MR devices can be used along with one or more microcoils or a plurality of microcoils can be used along with one or more MR devices to form a transponder 220.

In some embodiments, the transponder 220 can be tracked also using a plurality of receivers. Accordingly, the tracking system 200 can comprise a plurality of receivers (as well as the plurality of transmitters) and the microcoil 222 can be selected to be a five degree of freedom (“5DOF”) sensor. Further, similar to the tracking system 200 described above, the MR device 224 can be employed to provide the roll information which is the missing degree of freedom not obtained by the 5DOF sensor. In yet other embodiments, the transponder 220 can be tracked against an array comprising at least one transmitter and at least one receiver.

The tracking system 200 described in various embodiments can be used as a part of a surgical navigation system. In these embodiments, the transponder 220 is adapted to be inserted inside the object to be tracked. The transponder 220 can be inserted into the body of a patient while one or more transmitters 215 and the RF driver 210 are placed outside the body.

An example of these embodiments is shown in FIG. 3, where an object 305 includes an elongated probe, for insertion into the body of a subject 310 positioned on a patient positioning system 312. A transponder 315 can be fixed to the probe so as to enable an externally located signal processing unit 318 to determine the position and orientation of a distal end of the probe. Alternatively, the object 305 can include an implant, and the transponder 315 is fixed in the implant so as to enable the signal processing unit 318 to determine the position and orientation of the implant within the body. Further, the transponder 315 may be fixed to other types of invasive tools, such as endoscopes, catheters and feeding tubes, as well as to other implantable devices, such as orthopedic implants.

An externally located radio frequency driver 320 sends a radio frequency (RF) signal, having a frequency in the kilohertz range, to the object to be tracked. The plurality of electromagnetic transmitters 325 can be positioned in fixed locations outside the body to produce electromagnetic fields at different, respective frequencies, typically in the kilohertz range. These fields induce a voltage in the microcoil 222 and the MR device 224 of the transponder 315, which depend on the spatial position and orientation of the microcoil 222 and the MR device 224 relative to the transmitters 325. The control unit 226 converts the voltages into high-frequency signals, which are transmitted by the control unit 226, in the form of output signal, to the externally-located signal processing unit 318. The signal processing unit 318 processes the output signal to determine the position and orientation coordinates of the transponder 315, for display and recording.

Typically, prior to performing a medical procedure, the image of the subject 310 can be captured using an imaging device 330 (such as an X-ray imaging device) and is displayed on a computer monitor. The transponder 315 is visible in the X-ray image, and the position of the transponder 315 in the image is registered with the respective position coordinates, as determined by the signal processing unit 318. During the medical procedure, the movement of the transponder 315 is tracked by the tracking system 335 and is used to update the position of the transponder 315 in the image on the computer monitor, using image processing techniques known in the art. The updated image can be used to achieve desired navigation of the object 305 during the medical procedure, without the need for repeated X-ray exposures during the medical procedure.

In the embodiments shown in FIG. 4, an exemplary method 400 for tracking an object is described. The method 400 comprises positioning a radio frequency (RF) driver to transmit an RF driving current to the MR device contained in the transponder, as shown in box 405. The method continues in box 410 by inserting a transponder in or on the object 305, where the transponder contains a microcoil and a MR device. Next, the method 400 includes driving a plurality of transmitters to generate electromagnetic fields at respective frequencies in a vicinity of the object to induce a voltage drop across the microcoil and the MR device, as shown in box 415. The method continues in box 420 when the transponder generates an output signal indicative of the voltage drop across the microcoil and the voltage drop across the MR device. Next, the output signal is transmitted from the transponder as shown in box 425. The method includes and receiving and processing the output signal at the signal processing unit 318 to determine coordinates of the object, as shown in box 430.

In some embodiments, the method 400 can also include inserting the transponder, together with the object, into the body of the subject. And positioning the plurality of the transmitters and the RF driver includes placing one or more transmitters and the RF driver outside the body.

In some configurations, the subject is placed in a magnetic field generated by situating a pad under the subject which contains the plurality of transmitters for generating the electromagnetic field. The plurality of transmitters can generate electromagnetic fields at different, respective frequencies. A reference electromagnetic field sensor (not shown) can be fixed relative to the subject, for example, by being taped to the back of the subject and the object with the transponder can be advanced into the body of the subject. The signals received from the transponder are conveyed to the signal processing unit, which analyzes the signals and then displays the results on a display. Using this method, the precise position and orientation of transponder, relative to the reference sensor, can be ascertained and visually displayed. Furthermore, the reference sensor may be used to correct for breathing motion or other movement in the subject. Thus, the acquired position and orientation of the object may be referenced to an organ structure and not to an absolute outside the reference frame, which is less significant.

As described herein, a microcoil is combined with a MR device to obtain a transponder. The MR device replaces a second microcoil typically employed in some conventional tracking systems, thereby eliminating the use of the second microcoil. An advantage associated with the MR device is its ability to be fabricated as a miniature device. Thus, replacing the second microcoil with a MR device smaller than the second microcoil reduces the space needed.

Further, the MR device and the microcoil can share a single pair of leads. Thus, using the MR device allows for a simplified guide wire fabrication as the number of leads employed in connecting two components is reduced by half. Thus, the use of the MR device in a transponder enables the transponder to obtain six degrees of freedom (“6DOF”) while reducing burden on resource or space.

The systems and methods for tracking an object described herein may be implemented in connection with different applications extended to other areas. For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The systems and methods can be used in tracking an object in obscure environment, which can be adapted to track the position of items other than medical devices in a variety of applications. That is, the tracking systems and methods may be used in other settings where the position of an object in an environment is unable to be accurately determined by visual inspection. For example, tracking technology may be used in forensic or security applications. Retail stores may use tracking technology to prevent theft of merchandise. Tracking systems are also often used in virtual reality systems or simulators. Accordingly, the tracking systems and methods are not limited to medical devices, but can be carried further and implemented in various forms and specifications.

In addition to any previously indicated modification, numerous other variations and alternative arrangements may be devised by those skilled in the art without departing from the spirit and scope of this description, and the appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner.

1. A position transponder configured to operate inside a body of a subject, the transponder comprising:
a sensor coil configured to sense a voltage drop when an electromagnetic field is applied to the body of the subject containing the transponder, the electromagnetic field being applied from a transmitter external to the body; and
a magnetoresistive device coupled in series to the sensor coil via a single twisted pair or a coaxial cable;
wherein the transponder transmits an output signal indicative of the position of the transponder within the body.

2. The transponder of claim 1, wherein the sensor coil senses the voltage drop in response to multiple electromagnetic fields from multiple transmitters when applied to the body in a vicinity of the transponder.

3. The transponder of claim 1, wherein the magnetoresistive device is adapted to sense the electromagnetic field at a direction substantially perpendicular to the axis of the sensor coil and thereby experience a voltage drop.

4. The transponder of claim 3, further comprising a control unit coupled to the sensor coil and the magnetoresistive device so as to generate an output signal indicative of the voltage drop induced at the sensor coil and the voltage drop induced at the magnetoresistive device, such that the output signal is indicative of coordinates of the transponder inside the body.

5. The transponder of claim 4, wherein the control unit is further configured to transmit the output signal, so that the output signal is received by a signal processing unit positioned outside the body for use in determining the coordinates.

6. The transponder of claim 1, wherein the sensor coil is a microcoil.

7. The transponder of claim 5, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

8. A position transponder configured to operate inside a body of a subject, the transponder comprising:
a sensor coil, coupled so that a voltage drop is induced in the sensor coil when one or more electromagnetic fields is applied to the body of the subject containing the transponder, the one or more electromagnetic fields being applied from one or more transmitters external to the body;
a magnetoresistive device coupled to the sensor coil in series via a single twisted pair or a coaxial cable, such that a voltage drop is induced in the magnetoresistive device responsive to the electromagnetic fields applied to the body; and
a control unit, coupled to the sensor coil and the magnetoresistive device so as to generate an output signal indicative of the voltage drop induced at the sensor coil and the voltage drop induced at the magnetoresistive device, such that the output signal is indicative of coordinates of the transponder inside the body.

9. The transponder of claim 8, wherein the magnetoresistive device is adapted to sense the electromagnetic field at a direction substantially perpendicular to the axis of the sensor coil.

10. The transponder of claim 8, wherein the control unit is further adapted to transmit the output signal, so that the output signal is received by a signal processing unit positioned outside the body for use in determining the coordinates.

11. The transponder of claim 10, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

12. The transponder of claim 11, wherein the sensor coil is a microcoil.

13. A tracking system for tracking an object, comprising:
a radio frequency driver transmitting a radiofrequency driving current at a first frequency to the object;
a plurality of transmitters adapted to generate electromagnetic fields at different respective frequencies, including a second frequency, located external to the object;
a transponder within the object, the transponder comprising:
a sensor coil, the sensor coil configured to sense a voltage drop in response to exposure to the electromagnetic fields;
a magnetoresistive device coupled to the sensor coil in series via a single twisted pair or a coaxial cable, such that the magnetoresistive device is adapted to sense the electromagnetic field at a direction substantially perpendicular to the axis of the sensor coil and thereby experience a voltage drop; and
a control unit coupled to the sensor coil and the magnetoresistive device, so as to generate an output signal indicative of the voltage drop induced at the sensor coil and the voltage drop induced at the magnetoresistive device; and
a signal processing unit separate from and coupled to the transponder, the signal processing unit adapted to receive the output signal transmitted by the control unit and responsive thereto to determine the coordinates of the object.

14. The tracking system of claim 13, wherein the sensor coil is a microcoil.

15. The tracking system of claim 13, wherein the output signal is analog.

16. The tracking system of claim 13, wherein the output signal is digital.

17. The tracking system of claim 13, wherein the object is a catheter or an endoscope.

18. The tracking system of claim 13, wherein the control unit is adapted to generate the output signal indicative of an amplitude of the voltage drop and a phase of the voltage drop, and wherein the signal processing unit is adapted to determine the coordinates and an orientation of the object, responsive to the amplitude and the phase of the voltage drop indicated by the output signal.

19. A method for tracking an object, comprising:
positioning a radio frequency (RF) driver to transmit an RF driving current at a first frequency, to the object located within a body of a subject;
coupling to the object a transponder comprising a sensor coil and a magnetoresistive device that are connected using a single twisted pair or a coaxial cable;
driving a plurality of transmitters external to the body to generate electromagnetic fields at respective frequencies in a vicinity of the object that induce a voltage drop across the sensor coil and the magnetoresistive device;
generating an output signal at the transponder indicative of the voltage drop across the sensor coil and the voltage drop across the magnetoresistive device;
transmitting the output signal from the transponder; and
receiving and processing the output signal to determine coordinates of the object within the body.

20. The method of claim 2019, wherein driving the plurality of transmitters comprises driving the plurality of transmitters to generate the electromagnetic fields at different respective frequencies including a second frequency.

21. The method of claim 20, further comprising inserting the transponder, together with the object, into the body of a subject.

22. The method of claim 20, wherein positioning the plurality of transmitters and the RF driver comprises placing the plurality of transmitters and the RF driver outside the body.

23. The method of claim 19, wherein the magnetoresistive device is a magnetoresistive sensor.
